(19) United States
(12) Patent Application Publication
(10) Pub. No.: US 2006/0282009 A1
(43) Pub. Date: Dec. 14, 2006

(54) Device for measuring physical properties of the tympanic membrane

(76) Inventors: Ake Oberg, Ljungsbro (SE); Anders Johansson, Norrkoping (SE); Hans Knutsson, Linkoping (SE); Magnus Borga, Linkoping (SE); Tomas Stromberg, Linkoping (SE); Mikael Sundberg, Linkoping (SE)

Correspondence Address: INTELLECTUAL PROPERTY GROUP;FREDRIKSON & BYRON, P.A., 200 SOUTH SIXTH STREET, SUITE 4000, MINNEAPOLIS, MN, 55402, US

(21) Appl. No.: 10/560,410

(22) PCT Filed: Jun. 11, 2004
(86) PCT No.: PCT/SE04/00907

§ 371 Date: May 18, 2006

(30) Foreign Application Priority Data
Jun. 13, 2003 (SE) 0301718-3

(51) Int. Cl. A61B 5/00

(52) U.S. Cl. 600/559

(57) ABSTRACT

Device for measuring physical properties of the tympanic membrane (TM), comprising an elongated probe (12) with a distal end (15) for inspection of the ear, wherein a plurality of optical fibres is arranged in said elongated probe. The plurality of fibres includes either a first set of fibres (21) for conveying light from a light source to said distal end of said probe and a second set of fibres (22) for conveying light reflected from the tympanic membrane in front of said distal end to a first detector means (23) or a set of fibres both for conveying light from a light source to said distal end of said probe and for conveying light reflected from the tympanic membrane in front of said distal end to a first detector means (23). Said first detector means (23) is designed for measuring the intensity of light reflected from the tympanic membrane. Method for measuring physical properties of the tympanic membrane (TM), including the following steps: a) illuminating the tympanic membrane with light from a light source, b) detecting light reflected from the tympanic membrane, and c) analysing the intensity at selected wavelengths or a spectrum of wavelengths.

### TECHNICAL FIELD

The invention relates to a device and a method for measuring physical properties in general and physical properties of human tissues in the ear. The device in accordance with the invention can be used in connection with a diagnosis of acute otitis media (AOM).

AOM is one of the most common infectious diseases of childhood. Incidence figures vary greatly in the current literature. This probably reflects different threshold through time for seeking medical attention for earache and different diagnostic criteria between researchers rather than a true difference in incidence. AOM can in general terms be defined as purulent inflammation in the middle ear which starts abruptly, is of short duration and can be clinically verified.

Antibiotics have for long been recommended in the treatment of AOM but indefinite diagnostic criteria, a high percentage of spontaneous healing and an increasing awareness of microbial resistance have led to revision of the therapeutic guidelines in several European countries

Myringotomy with demonstration of purulent middle ear fluid, as a proof of bacterial infection, is considered the gold standard of AOM identification. In practice however, the diagnosis is often based on the combination of symptoms, such as earache, rubbing of the ear, fever, and changes of the characteristics of tympanic membrane (TM). An otoscopic assessment of the TM can be challenging even for the most experienced clinician because of overlapping findings with other conditions where antibiotics are not needed. Bulging due to the presence of middle ear fluid with decreased mobility and reddening and thickening of the TM with loss of the normal contour are signs associated with AOM but may also be seen in otitis media with effusion (OME). OME can be regarded as either a sequel of AOM or as a consequence of Eustachian tube dysfunction, and is characterised by the presence of a middle ear effusion for 3 months or more but a general absence of gross signs of infection. Redness of the TM can also be seen in virus related conditions such as common cold.

### PRIOR ART

Several studies of the otoscopic findings in AOM have failed to identify a specific sign or symptom in making an accurate diagnosis, but bulging of the TM seems to be an important variable. Pneumatic otoscopy, otomicroscopy, tympanometry and acoustic reflectometry are other previously suggested techniques for evaluating the TM as adjunctive tools in AOM diagnosis.

Fluorescence spectroscopy has been utilized by Sorrel et al Bacteria identification of otitis media with fluorescence spectroscopy, Lasers in surgery and medicine 1994;14:155-163, and Spector et al, Noninvasive fluorescent identification of bacteria causing acute otitis media in a chinchilla model. The Laryngoscope 2000;110:1119-1123, for the identification of pathogens causing AOM in vitro and in vivo.

### SUMMARY OF THE INVENTION

The inventors have assumed that the optical properties of the TM are similar to those of human skin since the TM is covered by epidermis lined by simple cuboidal epithelium. Consequently, the reflectance spectra of the healthy and the erythematous TM ought to differ in the same way as the spectra of healthy and erythematous human skin.

An object of the invention is to provide a device that will allow application of diffuse reflectance spectroscopy to perform a diagnosis of acute otitis media. The device in accordance with the invention comprises an elongated probe, a first end of which being operatively connected to a housing and a second end of which is designed to be inserted in the external auditory canal to a position close to the TM.

In the housing there is provided light generating means and at least one detector means. The light generating means are operatively connected to a plurality of optical fibers that extend through the probe to a position in the vicinity of the second end of the probe. The optical fibers normally are divided into at least two sets of fibers. A first set of the optical fibers is used to convey light from the light sources to the TM. A second set of fibres is used to convey light reflected from the TM to a photodetector arranged in the housing. Said second set of fibres can be divided further into subsets if different detectors are used. It is also possible to use the same set of fibers for conveying light in both directions, for instance by using so called fiber couplers.

The light from the light sources is directed towards the tissue in front of the probe and is used for diffuse reflectance spectroscopy. A minor portion of the light is specularly reflected from the surface and will have basically the same properties as the generated light. A major part of the light will penetrate into the tissue and interact with different objects such as red blood cells. The light reflected will be diffuse and due to different properties of the objects also properties of the light will change. The diffuse reflected light will have different intensities at different wavelengths.

The detecting means is arranged to receive the reflected light and to detect intensities at different wavelengths. In a first embodiment the detecting means comprises separate sensors for detecting different wavelengths. In a second embodiment reflected light is received in a single detector and then analyzed with regard to intensity at different wavelengths. It is possible also to use a combination of the detector embodiments.

In accordance with the invention a two parallel fibre array sensor can be designed to assess surface shapes of diffusely scattering media, without contact. Images are created by sequentially illuminating objects using one fibre array and detecting the diffusely back-scattered photons by the other array.

A separate set of fibres can be used to direct light from a plurality of separately controlled light sources to the tissue and to direct reflected light to a separate sensor for surface shape recognition of the TM. The separately controlled light sources are operated in sequence and light diffusely reflected from the TM is received by a plurality of sensor elements in the sensor for surface shape recognition. By combining the results of the diffuse reflectance spectroscopy detectors with the surface shape recognition sensors characteristic physical data of the TM can be obtained. The obtained data can be used to facilitate a diagnosis of AOM.

### BRIEF DESCRIPTION OF THE DRAWINGS

FIG. 1 is a schematic side elevational view of a first embodiment of a device in accordance with the invention including a control apparatus and a probe,

FIG. 2 is a schematic view showing the control apparatus of FIG. 1,

FIG. 3 is a cross sectional view from III-III in FIG. 1 of a first configuration of optical fibres In the tip of the probe,

FIG. 4 is a cross sectional view from III-III in FIG. 1 of a second configuration of optical fibres in the tip of the probe,

FIG. 5 is a cross sectional view from III-III in FIG. 1 of a third configuration of optical fibres in the tip of the probe,

FIG. 6 is a cross sectional view from III-III of a fourth configuration of the optical fibres In the tip of the probe, and

FIG. 7 is a schematic side elevational view of a second embodiment of a device in accordance with the invention including a probe,

FIG. 8A-C are illustrations of the surface area (A_{L}) illuminated by the emitting fibre and the area seen by the detecting fibre (A_{D}) for flat and convex surfaces.

### DETAILED DESCRIPTION

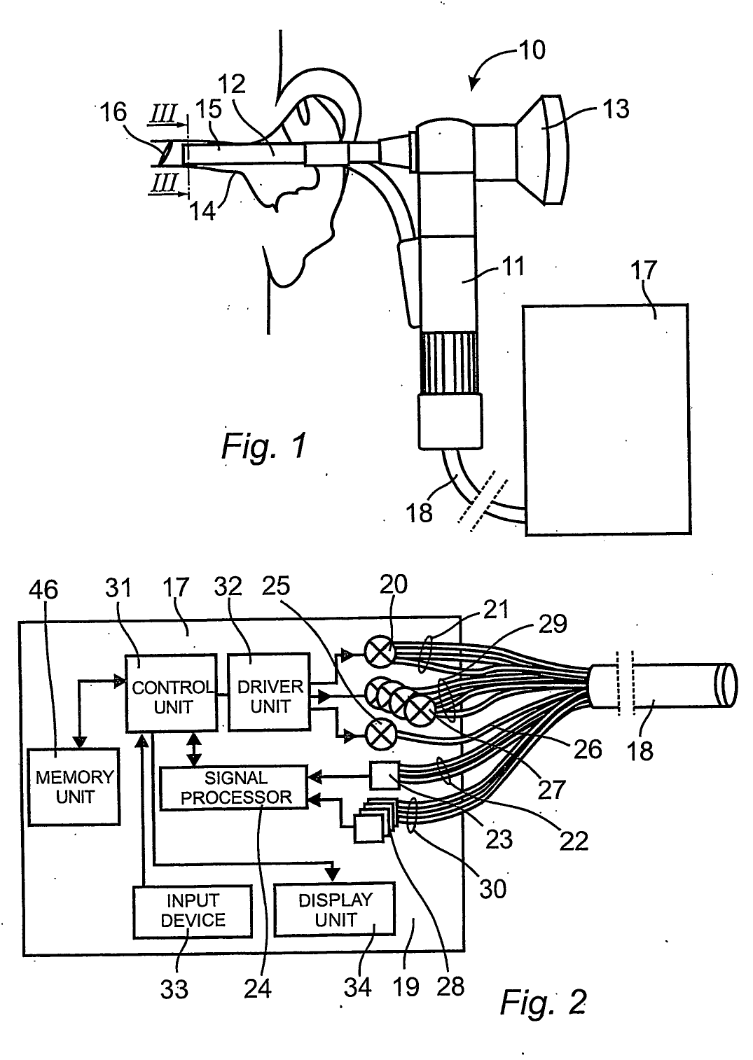

In the embodiment shown in FIG. 1 a device in accordance with the invention comprises an instrument 10 designed as a modified sinuscope suitable for visual inspection of narrow body cavities, such as the auditory canal. The instrument 10 is T-shaped with a vertical grip section 11 supporting a probe 12 and an eyepiece 13 extending in opposite directions. In FIG. 1 the probe is inserted in the external auditory canal 14. A tip 15 of the probe 12 is positioned 5-10 mm from the tympanic membrane 16.

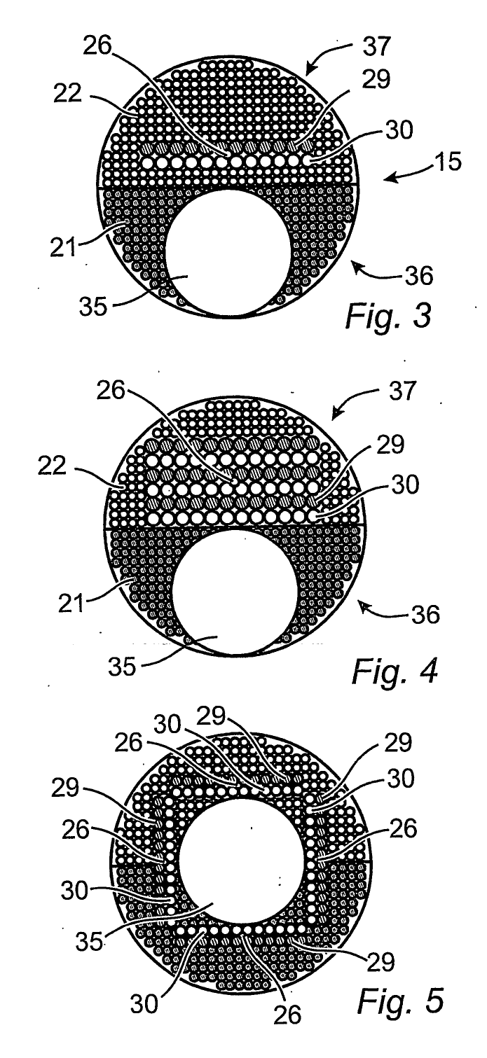

The instrument 10 is operatively connected to a control apparatus 17 through a cable 18. The cable 18 holds a plurality of optical fibres as will be described below. The optical fibres extend from a lower section of the vertical grip section to the probe 12. In the probe the optical fibres extend together with an ocular channel (c.f. FIG. 3-FIG. 5) that connects to the eye-piece 13.

The basic units of the control apparatus 17 are shown in FIG. 2. In this embodiment all units are enclosed in a cover 19. In other embodiments some or all units can be arranged as separate units or be provided in a computer and software implementation. A first light source 20 generates white light that is used for illuminating the TM. The light source serves both the visual inspection via the otoscope and as light for the diffuse reflectance spectroscopy as will be described below. The first light source can be similar to an Avantes HL-2000-LL, 7 W output, VIS-NIR spectral range, Eerbeek, Netherlands). Light from the first light source is directed into a first set of optical fibres 21 that is embedded in said cable 18 and extends to the end of the probe 12. The fibres in said first set of optical fibres are distributed in the end of the probe to provide an appropriate intensity level and a suitable distribution of light over the TM.

The light from the first set of optical fibres 21 is reflected from the TM and received in a second set of optical fibres 22 that extends also from the tip of the probe to the control apparatus 17. The fibres in the second set of optical fibres 22 are connected to a first detector means 23 that can be configured basically in different ways.

In a first embodiment the first detector means 23 is a single detector that is connected to a signal processor 24 in the control apparatus 17. The single detector produces data corresponding to the intensity of the diffuse reflected light. The signal processor 24 in this embodiment is configured to apply an erythema detection algorithm on the acquired data. A novel algorithm utilizes the fact that the photon absorption in the Q-band of various blood chromophores is different in erythymatous and in normal tissue.

A quantity, derived from the spectra, to be used for separating the states “erythematous tissue” and “normal tissue” that is independent of the geometrical distance between the probe head and site of measurement was desirable. For this reason the quotient

$${Q}_{λ} = \frac{{R}_{λ}}{{R}_{650}} \qquad ( 1 )$$

was used.
R₆₅₀ and R_{λ} are the reflectivity at 650 nm and λ nm, respectively. Normalization was performed by dividing every sample in each spectrum with its reflectivity at 650 nm. A variety of λ:s were tested. λ:s were selected in the absorption peak of bilirubin and the Q-band of oxyhemoglobin (HbO₂) (460 nm, 542 nm and 576 nm). In addition, λ-values were chosen based on measurements of Q_{λ} in normal and erythematous TM, in order to maximize discrimination. It was observed that Q_{λ} discriminated well at λ:s near 490 nm and 576 nm.

In accordance with the invention based on a two-wavelength or four-wavelength system the first detector means can include discrete detectors for each specific frequency. Each detector can be combined with a narrow filter, to achieve the desired frequency characteristics. Appropriate centre wavelengths are 460 nm, 490 nm, 542 nm, 576 nm and 650 nm. The detectors are connected to the signal processor 24 in the control apparatus 17. In such an embodiment the signal processor 24 can have a less complicated design.

In the embodiment shown in FIG. 2 a second light source 25 is also included. The second light source emits light that is directed towards the target tissue as a visual reference when the probe is positioned in the external auditory canal. A separate optical fibre, or a set of fibres, 26 is provided for conveying the light to the end of the probe. In one embodiment the second light source 25 is a laser diode that emits light at the wavelength 632 nm. In another embodiment as shown in FIG. 5 two separate optical fibres are used for determining the distance between the probe and the tympanic membrane and also for localising the probe in relation to the TM.

If high sensitivity and specificity are desired it may be appropriate not to rely on one single diagnostic parameter. Therefore, information about the color of the TM obtained as described above can be combined with other diagnostic parameters characterizing AOM, such as information about the geometry of the tympanic membrane as described below, still with reference to FIG. 2.

A third light source 27 can be provided for generating light that can be used in a surface shape recognition process. The third light source 27 comprises a plurality of individually controlled light emitting elements, such as light diodes (LED). Preferably, these diodes operate at a frequency such that the reflectivity will not be affected by the blood content of the tissue. A suitable λ is 650 nm. The light emitting elements are part of a fibre guided imaging system comprising in one embodiment 15 light emitting diodes and 15 photodiodes. The photodiodes form a second detector means 28.

Light from the light emitting elements 27 are conveyed to the probe end through the probe in a third set 29 of optical fibres, and reflected light is conveyed to the second detector means 28 by a fourth set 30 of optical fibres. All fibres are gathered in the cable 18.

Images are created from continuous detection of diffusely reflected photons when sequentially activating the light emitting elements 27. Fibers in the probe end are equidistant distributed in two parallel or concentrically arranged arrays, serving illumination and detection respectively. The spacing between the two fiber arrays is preferably small (500 μm or less). The radius of a spherical surface can be estimated by fitting an image, generated by an imaging system, with a theoretically generated image using the radius of the simulated surface as fitting parameter.

Signals from the first detector means 23 and the second detector means 28 are fed to the signal processor 24 forming part of an imaging system. The resolution of the generated images is highly dependent of the probe-surface distance and the numerical aperture (NA) of the fibers used. In one embodiment commercially available plastic fibers (NA=0.5) are used. The fibers can be arranged in two linear arrays in the probe head (one detector array and one illumination array), c.f. FIG. 3-FIG. 6.

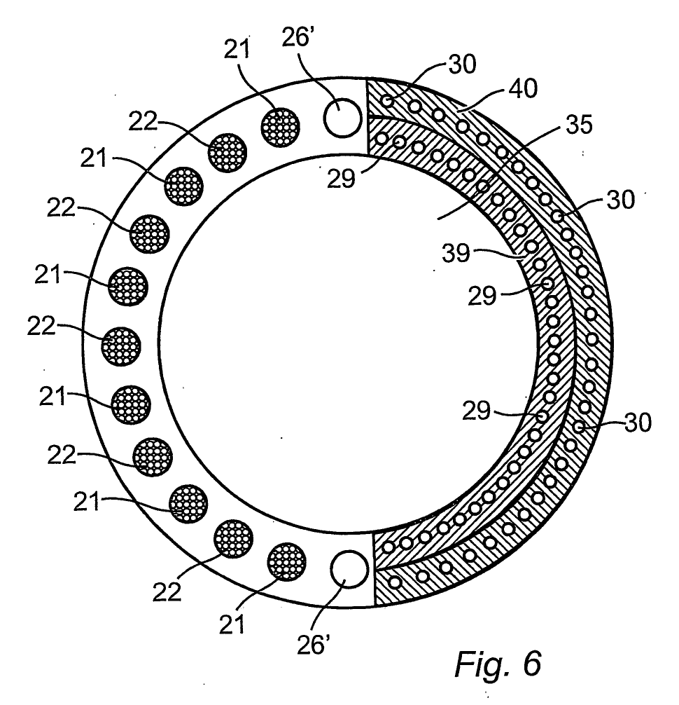

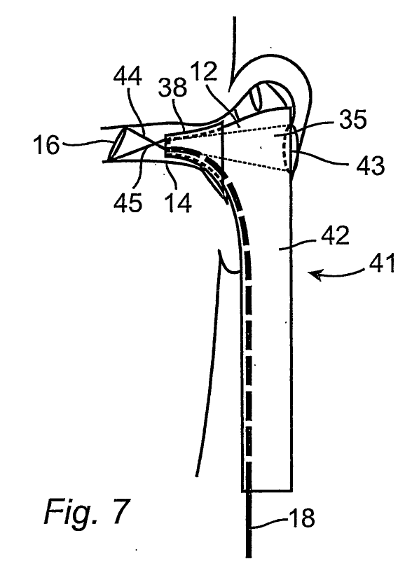

An appropriate mathematical model stipulates diffusely reflected photon detection. For this reason Polaroid filters can be appended in front of both the detector fiber array and the illumination fiber array, perpendicularly, to avoid detection of specularly reflected photons (c.f. FIG. 7). An example of filters is shown with reference to FIG. 6. The illumination and detector fibers are arranged in parallel and equidistantly distributed linearly in the lateral direction.

The number of optical fibres in the third set 29 and the fourth set 30 of optical fibres can be different from what is shown in the drawings and do not have to be equal.

Experimental data from convex and concave polyacetal plastic surfaces are recorded in a first step. A mathematical model of the sensor can also be used for simulating images of the surfaces analysed. The detected image is compared in a second step with the recorded data and a shape associated to the recorded data that corresponds best to the detected image is selected. An estimate of the shape characteristics of a surface is extractable from the images generated by the system. In particular, the system distinguishes perfectly accurate between convex and concave surfaces; which, e.g. is important when characterizing the TM.

The control apparatus 17 also comprises a control unit 31 operatively connected to other units of the control apparatus, such as the signal processor 24 and a memory unit 46. Experimental data or data created from the mathematical model is also stored in the memory unit. The light sources are driven by a driver unit 32, which is operated by the control unit. Data, such as operating commands, can be fed in by an input device 33, such as a keyboard or other appropriate means. In a simple embodiment the input device comprises a single trigger that will operate the control apparatus 17 when set into different positions. The trigger or any other suitable input device can be arranged on the instrument 10, for instance on the vertical grip section 11.

Data produced by the signal processor 24 and the imaging system can be displayed on a display unit 34, which also may include or consist of other audiovisual means, such as light diodes and loudspeakers. The data can also be transferred to further computing, analyzing and monitoring means (not shown). In one embodiment the display unit 34 is arranged to display an indication of the physical status of the TM. In a further developed system in accordance with the invention the display indicates the medical status of the TM. As stated above several units of the control apparatus 17, such as the control unit 31, the input device 33 and the display unit 34, can be part of a conventional personal computer or an application specific computer. The tip 15 of the probe is covered by a protective and optically neutral cap 38, c.f. FIG. 7. Preferably the cap 38 is disposable.

In the embodiments shown in FIG. 3 to FIG. 5 the tip 15 of the probe comprises a plurality of optical fibres and an ocular channel 35. The fibres are gathered in two semicircular sections. A first section 36 holds the first set 21 of optical fibres that is used for illumination. The ocular channel 35 also is arranged in the first section 36.

In a second semicircular section 37 of the tip 15 the second set 22 of optical fibres is provided. The number of individual fibres is chosen so as to supply each of the detectors in the first detector means 23 with a sufficient amount of reflected light. Normally, at least five individual fibres are used for each detector and each detector frequency. The separate optical fibre 26 is arranged in the second semicircular section 37 in the centre of a composite array formed by the third set 29 of optical fibres and the fourth set 30 of optical fibres that are used during surface shape recognition.

As shown in FIG. 3 the fibres in the third set 29 and the fourth set 30 of optical fibres are equidistantly distributed in two parallel arrays, serving illumination and detection, respectively. Preferably the spacing between the two fibre arrays is small, that is about 500 μm.

A first alternative embodiment of the tip end is shown in FIG. 4. Also in this embodiment the fibres are gathered in two semicircular sections. A first section 36 holds the first set 21 of optical fibres that is used for illumination. The ocular channel 35 also is arranged in the first section 36.

In a second semicircular section 37 of the tip 15 the second set 22 of optical fibres is provided. The number of individual fibres is chosen so as to supply each of the detectors in the first detector means 23 with a sufficient amount of reflected light. Normally, at least five individual fibres are used for each detector and each detector frequency.

In contrast to the embodiment of FIG. 3 three composite arrays formed by the third set 29 of optical fibres and the fourth set 30 of optical fibres are used. The arrays are disposed in close relationship and hold in a central position the separate optical fibre 26 that conveys light for a visual reference when the probe is positioned in the external auditory canal.

In a second alternative embodiment as shown in FIG. 5 four composite arrays formed by the third set 29 of optical fibres and the fourth set 30 of optical fibres are used. The arrays are disposed as four sides of a rectangle. In the centre of each of the arrays a separate optical fibre 26 is provided. The plurality of optical fibres 26 is optional, one fibre 26 is sufficient in this embodiment. When arranging several optical fibres 26, for instance as shown in FIG. 5, each of the fibres 26 can be positioned to emit light at a different angle to the perpendicular of the probe end surface. By such an arrangement it is possible to determine the distance between the probe end and the measuring object, in this case the TM. The ocular channel 35 in this embodiment is arranged in the centre of the probe end.

Light from the third set of optical fibres 29 will illuminate the surface along a line, each of the light emitting diodes 27 being turned on at a time. The sequence in which the light emitting diodes 27 are illuminated can be circular queue, such as a Round Robin scheduling algorithm, starting with activating a first fibre in the third set of optical fibres, continuing with the second, third and eventually the fifteenth followed by a restart of the sequence. As a result, a plurality of samples per detection fibre will be obtained in each time quantum of the illumination sequence. One measurement can extend over 100 cycles of each measurement, producing 100 images of a resolution of 15×15 pixels in the shown embodiment. Optimization to achieve real time image acquisition is possible.

The detecting fibres 30 and the corresponding second detector means 28 will be responsive to the light reflected from the surface and will produce a signal that will indicate the curvature of the surface. A set of measurements is made in advance on a plurality of standard shaped bodies having different and specified concave and convex shape. The results of the measurements are stored in the memory unit 46. A specific curvature is determined by comparing the detected image data with previously stored data and electing the curvature that presents the best conformity with the stored data.

To compensate for system dynamics it is possible to normalize the images acquired from curved surfaces by using an image acquired from a flat surface. This can be done by dividing each image element with the corresponding element in the image of the flat surface.

In FIG. 6 an embodiment comprising an annular configuration of the fibre carrying part of the probe head is shown. A left half of the probe head is used for the first set of optical fibres 21 and the second set of optical fibres 22, while a right hand side of the probe head carries the third set of optical fibres 29 used for illumination and the fourth set of optical fibres 30 used for curvature recognition. A central part of the probe head forms the ocular channel 35.

On the left hand side a plurality of channels are formed in the probe head and in each channel a plurality of fibres are arranged. Every second channel holds elements of the first set of optical fibres 21 and every second channel holds elements of the second set of optical fibres 22. These fibres and the corresponding detector means are operated in correspondence with the description with reference to FIG. 3-FIG. 5. All sets of fibres are arranged along a semicircular line outside the ocular channel 35.

Two separate optical fibres, or set of fibres, 26′ arranged opposite each other are provided for facilitating the positioning of the probe head in the ear of a patient. In this embodiment the second light source 25 produces a collimated light that will be directed from the optical fibres 26′ in two intersecting beams (c.f. FIG. 7). After intersecting the light beams will hit the tympanic membrane in two separate and distinctive positions. By adjusting the distance between the probe and the tympanic membrane until target areas of the light beams are located at opposite side edges of the tympanic membrane it is possible position the probe at an appropriate distance from the tympanic membrane.

On the right hand side of the probe head a plurality of channels are formed in two concentric lines. The channels in an inner line hold the third set of optical fibres 29 that are used for illuminating the tympanic membrane in the curvature recognition process. The channels in an outer line hold the fourth set of optical fibres 30 that are used for detecting the curvature of the tympanic membrane. The third set of optical fibres 29 is arranged and positioned to direct emitted light to a straight line on a flat surface. By illuminating each of the fibres in the third set of optical fibres 29 in sequence, for instance as described above, an image indicative of the curvature of the tympanic membrane can be obtained from the fourth set of optical fibres 30.

A first annular section, carrying the illuminating third set of optical fibres 29, is covered by a first polarisation filter 39 and a second annular section, carrying the fourth set of optical detection fibres 30 is covered by a second polarisation filter 40. The direction of polarisation of the first filter was rotated 90° relative to the second, to assure maximum attenuation of specularly reflected photons.

FIG. 7 shows a schematic view of a second embodiment of the instrument 41 in accordance with the invention. The instrument 41 is compact and comprises an integral probe 12. A vertical grip section 42 is also integral with the probe and a lens 43 replaces the eyepiece in the previous embodiment. The probe 12 is inserted in the external auditory canal 14 the ocular channel 35 provides a possibility for an operator of the instrument to observe through the lens 43 the status of the tympanic membrane 16 and to perform measuring process with the instrument.

The positioning of the probe is facilitated by a first beam 44 and a second beam 45 emitted from the separate optical fibres 26′. The instrument 10 is in an appropriate position when the first beam 44 and the second beam 45, respectively, strike opposite end portions of the tympanic membrane 16 as shown in FIG. 7. The tip 15 of the probe is protected by a protective and optically neutral cap 38 as schematically shown.

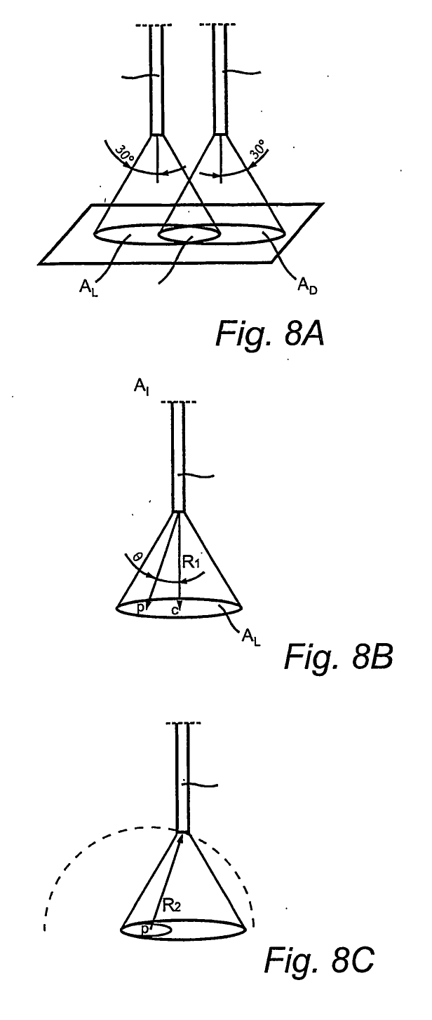

In a theoretical model as illustrated in FIG. 8A to FIG. 8C it is assumed that the detectable light-intensity signal I_{C} from an illuminated surface originates from photons backscattered from the intersection A_{I} in between the illuminated surface A_{L}, and the surface seen by the detector A_{D}, c.f. FIG. 8A. The intensity of the illuminated surface is governed by the inverse square law and the spatial distribution of the illumination I_{P} from the fibre was assumed to equal to an ideal Lambert source, c.f. FIG. 8B and equation (2).

$${I}_{c} ∝ \frac{1}{{R}_{1}^{2}} \\ {I}_{p} ∝ {I}_{c} cos θ \qquad ( 2 )$$

Photons that are back-scattered, from the turbid medium, were assumed to exit the medium in random directions (diffuse scattering) and to be detectable if exiting the medium from A_{I} in a direction within the acceptance angle of the detector fibre. The detectable fraction of the back-scattered intensity I_{D} was also assumed governed by the inverse square law, c.f. FIG. 8C and equation (3).

$${I}_{D} ∝ {I}_{p} \frac{1}{{R}_{2}^{2}} \qquad ( 3 )$$

An example of surface characteristics identification can be presented if considering convex and concave spherical surfaces. Such surfaces are characterised by the radius of the curvature. In the convex case, the probe can be considered to be localised outside the sphere, and in the concave case inside the sphere. As an example of a surface curvature classification algorithm, the difference between the mean of the diagonal elements and the mean of the fifth off-diagonal elements of the image generated by the two-parallel-fibre-sensor can be used. In the convex case, the difference is positive; contrary from the concave case, where it is negative. For a plane surface the difference is zero or close to zero. The radius of curvature can be extracted by empirical or theoretical matching of the image generated by the sensor with images from the same class of surfaces (i.e. images of convex or concave surfaces with different curvature radii in the range of interest).

1. Device for measuring physical properties of the tympanic membrane (TM), comprising an elongated probe (12) with a distal end (15) for inspection of the ear, wherein a plurality of optical fibres is arranged in said elongated probe characterised in that the plurality of fibres includes either a first set of fibres (21) for conveying light from a light source to said distal end of said probe and a second set of fibres (22) for conveying light reflected from the tympanic membrane in front of said distal end to a first detector means (23), or a set of fibres both for conveying light from a light source to said distal end of said probe and for conveying light reflected from the tympanic membrane in front of said distal end to a first detector means (23), that said first detector means (23) is designed for measuring the intensity of light reflected from the tympanic membrane.

2. Device in accordance with claim 1, wherein said first detector means (23) is a single detector for detecting the light intensity at selected wavelengths or at a spectrum of wavelengths, that is connected to a signal processor (24) provided in a control apparatus (17), said signal processor (24) being configured to apply an erythema detection algorithm on data acquired from said first detector means (23).

3. Device in accordance with claim 2, wherein said erythema detection algorithm utilizes the fact that the photon absorption in the vicinity of the Soret band and the Q band of various blood chromophores is different in erythematous and in normal tissue.

4. Device in accordance with claim 1, wherein said first detector means (23) comprises at least two separate detectors, a first detector having a peak sensitivity at 650 nm and a second detector having a peak sensitivity at 576 nm.

5. Device in accordance with claim 4, wherein said first detector means (23) comprises at five separate detectors, a first detector having a peak sensitivity around 650 nm, a second detector having a peak sensitivity around 460 nm, a third detector having a peak sensitivity around 490 nm, a fourth detector having a peak sensitivity around 542 nm, and a fifth detector having a peak sensitivity around 576 nm.

6. Device in accordance with claim 1, wherein the plurality of fibres includes a first set of illumination fibres (29), each of said illumination fibres being connected in a first end to one of a plurality of individually controllable light sources (27), and a second set of detecting fibres (30), said second set of detecting fibres being connected in a first end to individual detectors (28), said first set of illumination fibres (29) and said second set of detecting fibres (30), wherein said individually controllable light sources (27) are connected to a control unit (31) arranged to switch on said individually controllable light sources (27) in a sequence and wherein said individual detectors (28) are connected to said signal processor (24) for conveying signals responsive to the intensity of incident light reflected from the tympanic membrane.

7. Device in accordance with claim 6 where first set of illumination fibres (29) and said second set of detecting fibres (30) are equidistantly distributed in two parallel or concentric arrays in the distal end (15), or where first set of illumination fibres (29) and said second set of detecting fibres (30) are inter-leaved at the distal end (15).

8. Device in accordance with claim 6, wherein said first set of illumination fibres (29) is arranged to direct emitted light in the form of a line on to a tar-get surface.

9. Device in accordance with claim 6, wherein a memory unit (46) is provided for storing signals responsive to the intensity of incident light reflected from a plurality of bodies having different and specified concave and convex surfaces together with the corresponding surface data, and wherein said control unit (31) is designed for comparing said stored signals with signals obtained from a tympanic membrane and electing the surface having a correspondence with the signals obtained from a tympanic membrane.

10. Device in accordance with claim 1, wherein said first set of fibres (21) for conveying light from a light source to said distal end of said probe and said second set of fibres (22) for conveying light reflected from the tympanic membrane in front of said distal end to a first detector means (23) are arranged along a circular line and wherein an ocular channel (35) is arranged radially within said circular line.

11. Device in accordance with claim 9, wherein a separate optical fibre, or set of fibres, (26′) is arranged on either side of said ocular channel (35) diametrically opposed to each other for directing light towards the tympanic membrane and for producing visual reference points on the tympanic membrane.

12. Device in accordance with claim 6, wherein said first set of fibres (21) is distributed in a first semicircular section (36) in the distal end (15) together with an ocular channel (35) and wherein said second set of fibres (22) is distributed in a second semicircular section (37) in the distal end (15) together with said first set of illumination fibres (29) and said second set of detecting fibres (30).

13. Device in accordance with claim 8, wherein a separate optical fibre, or set of fibres, (26, 26′) is operatively connected to a second light source (25) for conveying light that is directed towards target tissue as a visual reference.

14. Device in accordance with claim 1, wherein said probe (12) extends from a vertical grip section (11) and an eyepiece (13) is optically connected to an ocular channel extending through said probe (12).

15. Method for measuring physical properties of the tympanic membrane (TM), including the following steps:
a) illuminating the tympanic membrane with light from a light source,
b) detecting light reflected from the tympanic membrane, and
c) analysing the intensity at selected wavelengths or a spectrum of wavelengths.

16. Method in accordance with claim 14, also including the following steps:
a) illuminating in sequence individual spots distributed over the tympanic membrane,
b) detecting the intensity of light reflected from the spots of the tympanic membrane and
c) determining the shape of the tympanic membrane by comparing said detected intensities with stored intensities obtained from type bodies having different shapes.
